(19) United States
(12) Patent Application Publication
(10) Pub. No.: US 2026/0263706 A1
(43) Pub. Date: Sep. 10, 2026

(54) TISSUE INJECTION DEVICE

(71) Applicant: BlueRock Therapeutics LP, Cambridge, MA (US)

(72) Inventors: Gregory Pollieri, Toronto (CA); Anna M Galea, Stow, MA (US); Dusan Zivkovic, Toronto (CA)

(73) Assignee: BlueRock Therapeutics LP, Cambridge, MA (US)

(21) Appl. No.: 19/165,523

(22) PCT Filed: Mar. 14, 2024
(86) PCT No.: PCT/US2024/019843

§ 371 Date: Sep. 15, 2025

Related U.S. Application Data
(60) Provisional application No. 63/491,043, filed on Mar. 17, 2023.

(51) Int. Cl. A61M 5/46; A61B 17/34; A61M 5/315; A61M 5/32

(52) U.S. Cl. CPC A61M 5/46; A61M 5/31586; A61M 5/3232; A61B 17/3478

(57) ABSTRACT

Embodiments disclosed herein relate to tissue injection devices for use in delivering therapeutic substances into tissue. A tissue injection device may comprise an actuator, an outer shaft, one or more contacts configured to contact the tissue, and an injection needle moveable within the outer shaft and configured to be inserted into the tissue to deliver therapeutic substances into the tissue. The actuator may be configured to actuate the injection needle between a retracted and an extended position.

### CROSS REFERENCE TO RELATED APPLICATION

This application claims the benefit of U.S. Provisional Application No. 63/491,043, filed Mar. 17, 2023, which is hereby incorporated by reference in its entirety.

### FIELD

Disclosed embodiments are generally related to injection devices for use in delivering therapeutic substances into tissue.

### BACKGROUND

A variety of methods have been employed to deliver therapeutic substances to patients including intravascular, intramuscular, subcutaneous, oral, and topical administration. Specifically, tissue injection devices have been employed for use in delivering therapeutic substances into a tissue of a patient. In some arrangements, an injection needle may be included in such devices, where the injection needle may be advanced into the tissue of the patient to permit delivery of the therapeutic substances.

### SUMMARY

According to one aspect of the invention, a tissue injection device is provided. In some embodiments, the tissue injection device may comprise an actuator, an outer shaft having proximal and distal portions as well as a shaft opening, one or more contacts configured to contact a tissue surface, and an injection needle moveable through at least a portion of the outer shaft. The one or more contacts may be moveable between a contracted and an expanded configuration. The actuator may be configured to actuate the injection needle between a retracted and an extended position.

According to another aspect of the invention, a tissue injection device is provided. In some embodiments, the tissue injection device may comprise an outer shaft having proximal and distal portions as well as a shaft opening, one or more contacts configured to contact a tissue surface, an injection needle moveable through at least a portion of the outer shaft, and at least one stop surface. The one or more contacts may be moveable between a contracted and an expanded configuration and may be biased towards the contracted configuration. The at least one stop surface may be disposed at the distal portion of the outer shaft and may be configured to abut the one or more contacts to restrict movement of the contacts beyond a predetermined position.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A depicts a tissue injection device according to some embodiments;

FIG. 1B depicts the tissue injection device shown in FIG. 1A, with the tissue injection device inserted through a chest wall and interacting with the heart wall;

FIG. 2A is an enlarged view of region 2A of FIG. 1A, depicting contacts in an expanded configuration;

FIG. 2B depicts the region shown in FIG. 2A, with an injection needle actuated into an extended position;

FIG. 2C depicts the region shown in FIG. 2A, with the injection needle inserted into tissue;

FIG. 3 depicts another embodiment of a tissue injection device;

FIG. 4A is an enlarged view of region 4A of FIG. 3, depicting contacts in a contracted configuration;

FIG. 4B depicts a portion of the region shown in FIG. 4A, with the contacts in an expanded configuration;

FIG. 4C depicts the region shown in FIG. 4A, with an injection needle actuated into an extended position;

FIG. 5 depicts another embodiment of a tissue injection device;

FIG. 6A is an enlarged view of region 6A of FIG. 5, depicting contacts in a contracted configuration;

FIG. 6B depicts the region shown in FIG. 6A, with the contacts in the expanded configuration;

FIG. 6C depicts the region shown in FIG. 6A, with an injection needle actuated into an extended position;

FIG. 7 depicts another embodiment of a tissue injection device;

FIG. 8A is an enlarged view of region 8A of FIG. 5, depicting contacts in a contracted configuration;

FIG. 8B depicts the region shown in FIG. 8A, with the contacts in an expanded configuration; and

FIG. 8C depicts the region shown in FIG. 8A, with an injection needle actuated into an extended position.

### DETAILED DESCRIPTION

It should be appreciated that aspects of the invention disclosed herein are not limited in application to the details of the construction and the arrangement of components set forth in the following description or illustrated in the drawings. Further, the concepts disclosed herein may be arranged in any suitable combination, as the present disclosure is not limited in this respect. Also, the terminology used herein is for the purpose of description and should not be regarded as limiting.

Tissue injection devices may be used to provide a targeted means of injecting therapeutic substances into tissue. In some embodiments, tissue injection devices may be used to provide injection into tissue internal to a patient (e.g. the myocardium). Some conventional tissue injection devices operate by inserting an injection instrument (e.g. an injection needle) into a target site of tissue, where the injection instrument may then deliver one or more therapeutic substances (e.g. drug products) into the tissue. The injection instrument may then be withdrawn from the injection site following use. However, the inventors have recognized that conventional tissue injection devices may have drawbacks including, but not limited to, potential damage to the tissue during operation (e.g. perforation of the myocardium), difficulty in positioning and/or stabilizing the injection device for insertion into the tissue, and/or a lack of precise control over the depth and/or angle of insertion into the tissue (e.g., when injecting into heart tissue while the heart is beating).

The inventors have recognized the need for tissue injection devices that provide improvements relative to some or all of the drawbacks disclosed above.

Some aspects disclosed herein relate to the use of an actuator in a tissue injection device to actuate an injection needle into a tissue of a patient, thereby permitting dispensing of therapeutic substances into the tissue. The inventors have recognized that the use of an actuator may allow for greater precision in controlling depth of insertion of the injection needle into the tissue. By comparison, certain alternatives such as disposable hypodermic syringes may not permit such precise injection. The ability to precisely control the depth of needle insertion may also reduce the risk of unwanted backflow of the therapeutic substances during injection by ensuring the needle is at the proper depth for injection into the target tissue.

Some aspects disclosed herein also relate to the use of one or more contacts provided in a tissue injection device, with the one or more contacts being configured to expand to contact a tissue surface. The inventors have appreciated that the use of contacts may provide a larger surface area contacting the tissue surface, thereby promoting stability and accurate positioning of the injection device when engaged with the tissue surface. In some embodiments, the target tissue site may be a moving surface, e.g. a beating heart. The contacts may help to promote engagement with the tissue surface even during movement of the tissue surface. The increased surface area of contact may help to decrease the amount of pressure subjected to the tissue surface, which may help to reduce the risk of trauma to the tissue. The one or more contacts may help to promote the proper insertion depth of the needle into the tissue. The risk of adverse health effects such as embolism due to improper injection may be reduced with use of the one or more contacts. The inventors have recognized that the contacts may conform to the contours of the tissue surface to permit stabilized injection into the tissue at any suitable angle and that the contacts may serve to limit damage to the tissue since the force applied to the tissue may be distributed over a larger contact area.

In some embodiments, a tissue injection device may include stop surfaces configured to restrict movement of the contacts past a predetermined position.

Aspects of tissue injection devices disclosed herein may be used in any suitable application for injection and delivery of therapeutic substances into a tissue of a patient. For example, potential applications include intracardiac injections, such as injection into the myocardium of a patient. In some such arrangements, the injection device may abut the outer surface of the heart wall (i.e. the epicardium) by extending through a hole in the chest wall, where an injection needle may then be deployed through the epicardium into the myocardium to dispense the therapeutic substances into the myocardium. While this specific arrangement is disclosed, the tissue injection device may be employed in any suitable configuration to deliver therapeutic substances into any suitable target tissue site. A therapeutic substance for injection with any of the devices described herein may comprise small molecules (i.e., chemical compounds), larger molecules (nucleic acids such as RNA or exosomes, peptides or proteins like antibodies), a gene therapy (e.g., a viral vector or an LNP-contained nucleic acid), cellular cells, a combination of any the aforementioned molecules or any other suitable form of therapeutic substance as detailed below.

In some embodiments, a tissue injection device may be used to deliver suitable therapeutic substances into tissue in the form of a cell delivery solution or matrix. A cell delivery solution may comprise any sort of cell. In some embodiments, a cell delivery solution comprises a human cell. In some embodiments, cells comprised in a cell delivery solution may be a stem cell or a stem cell derivative, e.g., a mesenchymal stem cell, umbilical cord-derived mesenchymal stem cell, cardiac stem cell, or hematopoietic stem cell. In some embodiments, cells comprised in a cell delivery solution may be a pluripotent cell (PSC) or a PSC-derived cell. A PSC may be an embryonic PSC or an induced PSC. In some embodiments, cells comprised in a cell delivery solution may be a bone marrow-derived mononuclear cell or a bone marrow-derived mesenchymal cell. A cell delivery solution may comprise an ectodermal cell, a mesodermal cell, or an endodermal cell, or a precursor or progenitor or derivative thereof. In some embodiments, a cell delivery solution comprises cardiac cells or cells of the heart. Cardiac cells may be vascular, fibroblastic, stromal, mesothelial, endothelial, immune in nature (e.g., a macrophage, a T cell such as a Treg cell or a killer T cell (e.g., CD8+ or CD4+)), a cardiomyocyte, a smooth muscle cell, a pericyte, or a progenitor or precursor of any thereof. In some embodiments, and without limitation, a cardiac cell is a cell of a ventricle (e.g., right or left ventricle), atrial (e.g., right or left atria), a node (e.g., sinoatrial node or atrioventricular node), vascular smooth muscle, endocardium, myocardium, epicardium, aorta, pulmonary trunk, or coronary artery. Other suitable, types of cells that may be delivered to a target tissue site include, but are not limited to: neural cells, neuron cells, mesenchymal stem cells, hematopoietic stem cells, embryonic stem cells or induced pluripotent stem cells, red blood cells, platelets, chondrocytes, skin cells, immune cells (e.g. tumor infiltrating lymphocytes, viral reconstitution T cells, dendritic cells, regulator T cells, macrophages), neural crest stem cells, neurons, glia, smooth muscle, cardiac tissue, chondrocytes, osteocytes, glial restricted progenitors, astrocytes, oligodendrocytes, neuroblast cells, megakaryoblasts, megakaryocytes, monoblasts, monocytes, macrophages, myeloid dendritic cells, proerythroblasts, erythroblasts, normoblasts, reticulocytes, thrombocytes, myeloblasts, progranulocytes, neutrophilic myelocytes, neutrophilic band cells, neutrophils, eosinophilic myelocytes, eosinophilic band cells, eosinophils, basophilic myelocytes, basophilic band cells, basophils, committed lymphoid projenitors, pre-NK cells, NK lymphoblasts, NK cells, thymocytes, T-lymphoblasts, T-cells, plasmacytoid dendritic cells, pre-B cells, B13 lymphoblasts, B cells, plasma cells, osteoblasts, myoblasts, myotubes, fibroblasts, adipocytes, mesoderm, ectoderms, primordial germ cells, sperm, eggs, definitive endoderm, or any other suitable type of cell. In some embodiments, a cell delivery solution comprises a precursor or progenitor or derivative cell of any of the aforementioned cells. In some embodiments, a cell is engineered (e.g., modification of the genome, or insertion of an expression vector or other nucleic acid).

In some embodiments, the tissue injection device may be used to obtain a tissue biopsy or non-tissue sample (e.g., an aspirate).

In some embodiments, the tissue injection device may include a device body and an outer shaft having a proximal and distal portion, with the proximal portion of the outer shaft being coupled to the device body. The distal portion of the outer shaft may further have a shaft opening.

In some embodiments, the tissue injection device may further comprise one or more contacts disposed on the distal portion of the outer shaft, with the contacts being moveable between a contracted configuration and an expanded configuration. In the expanded configuration, the contacts may have a larger surface area configured to abut a tissue surface and to stabilize the injection device relative to the tissue surface. The one or more contacts may be actuated between configurations using a variety of suitable methods, as is discussed in further detail below.

In some embodiments, the tissue injection device may include an injection needle, and the needle may be moveable through at least a portion of the outer shaft. The injection needle may be configured to move between a retracted position and an extended position, where the extended position may extend past the shaft opening at the distal portion of the outer shaft to deploy into a target tissue site. In some such arrangements, suitable therapeutic substances may then be dispensed out of the needle as disclosed herein.

According to one aspect, a tissue injection device may include an actuator configured to actuate the injection needle. The actuator may be configured to move the injection needle between the retracted position and the extended position such that the actuator may deploy the injection needle into a tissue of a patient for use in dispensing the therapeutic substances.

In some embodiments, the device body may include a handle configured for use as a handheld tissue injection device. In some embodiments, the actuator may comprise a trigger. However, it should be appreciated that the actuator may be any suitable arrangement, such as a button that is depressed by a user, a rotary knob, a sliding actuator, etc. The use of an actuator may serve to provide a controlled depth of needle insertion into the target tissue site.

In some embodiments, the actuator may also permit movement of the one or more contacts disposed at a distal portion of the outer shaft between the contracted and expanded configurations. In some embodiments, the actuator may serve to actuate both the injection needle and the one or more contacts. In some such arrangements, the actuator may function as a two-stage actuator, where the first stage includes actuating the contacts between the contracted and expanded configurations, and the second stage includes actuating the injection needle between the retracted and extended positions. While this specific arrangement is disclosed, the actuator may be arranged to actuate one or more components of the tissue injection device in any suitable manner as the disclosure is not so limited. For example, the actuator may actuate both the one or more contacts and the injection needle simultaneously, or the actuator may actuate the injection needle prior to actuation of the one or more contacts. In some embodiments, the actuator may actuate only the needle or only the contacts, and not both.

In some embodiments, the needle may include one or more outlet openings through which a therapeutic substance is released in order to introduce the substance into a target tissue site. The needle may be hollow such that the therapeutic substance passes through a lumen of the needle and out through the outlet openings. In some embodiments, the needle may be of a suitable shape including, but not limited to straight, curved, and helical. In some embodiments, the needle may have side ports.

In some embodiments, the tissue injection device may include at least one fluid fitting such as a luer fitting to permit fluid communication with the lumen of the needle. In such a configuration, the luer fitting may serve as an inlet to receive one or more therapeutic substances from one or more therapeutic substance storage components, and the luer fitting may have features to permit coupling of the luer fitting with such a storage component. For example, a syringe comprising a therapeutic substance may be connected to a luer fitting that is in fluid communication with a lumen of a needle. The syringe may then inject the therapeutic substance through the luer fitting into the lumen of the needle, thereby permitting delivery of the therapeutic substance into the target tissue site through the outlet opening(s) of the needle. In some such arrangements, the tissue injection device may function as a two-handed system, where the user may use the actuator to deploy the needle into an extended position and also use the luer fitting and the corresponding therapeutic substance storage component to supply the therapeutic substance to the target tissue site. While these specific configurations are disclosed, the therapeutic substance may be supplied to the target tissue site using a variety of suitable modes including, but not limited to, manually (e.g. with syringes), electromechanically, mechanically, or any other suitable mode.

Turning to the figures of the present invention, specific non-limiting embodiments of the invention are described in further detail. It should be understood that the various systems, components, features, and methods described relative to these embodiments may be used either individually and/or in any desired combination as the disclosure is not limited to only the specific embodiments described herein.

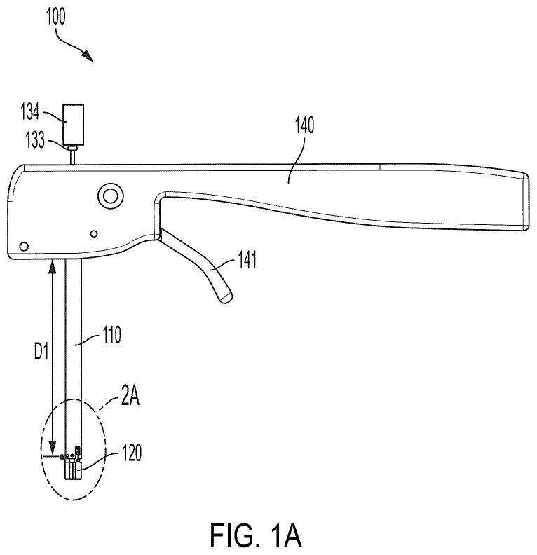

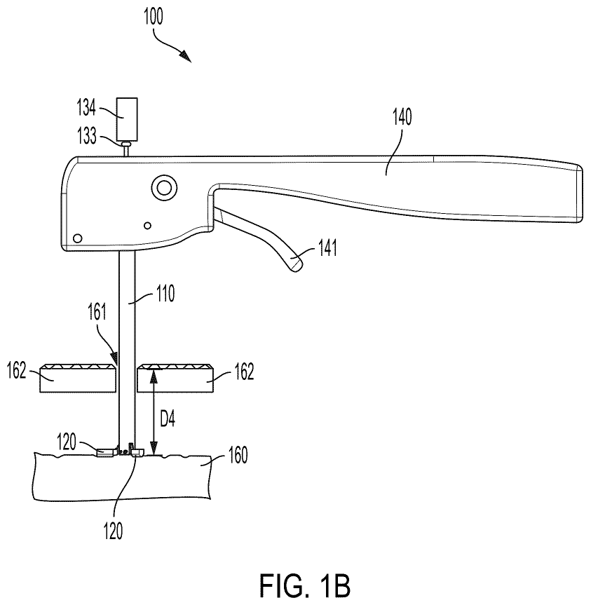

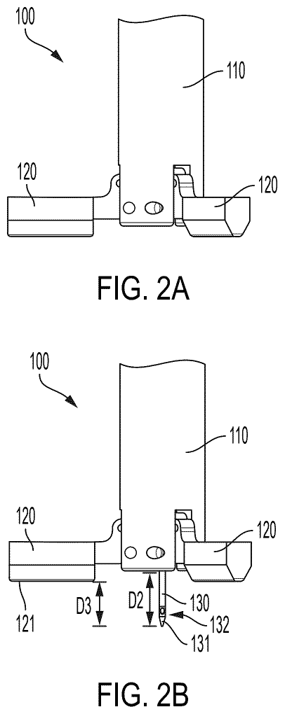

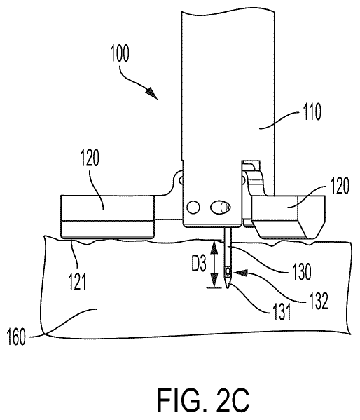

The illustrative embodiment of FIG. 1A shows a tissue injection device 100 comprising an outer shaft 110, contacts 120 disposed at the distal portion of the outer shaft, a device body 140 in the form of a handle connected to the proximal portion of the outer shaft, and an actuator 141 comprising a trigger. The actuator 141 may be configured to actuate the contacts 120 and/or an injection needle (not pictured) which may be at moveable within at least a portion of the outer shaft. The contacts 120 of the present embodiment are shown in the contracted configuration, which may be actuated into the expanded configuration via the actuator 141. In the contracted configuration, the contacts 120 may be of a smaller transverse dimension relative to the expanded configuration such that the transverse dimension of the contacts is of a similar or smaller size to the diameter of the outer shaft 110. The smaller transverse dimension of the contracted configuration of the contacts may allow for ease of entry of the distal portion of the tissue injection device into the body in order to reach the target tissue site. The outer shaft 110 and the contacts 120 may be configured to be inserted through an opening (e.g. a hole in the chest wall) and moved toward the target tissue site internal to the patient (e.g. the myocardium). The tissue injection device 100 also includes an inlet 133, such as a luer fitting, configured to receive therapeutic substances into the device. The inlet 133 may be in fluid communication with a lumen of an injection needle (see, e.g., injection needle 130 in FIG. 2B) of the device. Various therapeutic substances disclosed above may be introduced into the inlet 133. In some embodiments, a storage container 134, e.g. a vial, syringe, or other suitable container, may be used to introduce a therapeutic substance into the inlet 133. The storage container 134 may be couplable with the inlet 133, directly or via connecting components such as tubing or other couplings.

FIG. 1B shows the tissue injection device 100 of FIG. 1A inserted through a hole 161 of a patient and interacting with a tissue surface 160 internal to the patient. The hole 161 is formed in an external tissue surface 162 of the patient. In some embodiments, the external tissue surface 162 is the chest wall and the internal tissue surface 160 is the heart tissue of a patient (i.e. epicardium). The contacts 120 of the tissue injection device 100 are shown in the expanded configuration in this embodiment for use in providing stability of the injection device 100 relative to the internal tissue surface 160. The hole 161 formed in the patient may be of any suitable size or shape to permit insertion of the contacts 120 and outer shaft 110 of the tissue injection device 100.

In some embodiments, a hole depth (see D4 in FIG. 1B) in a patient for insertion of the contacts and outer shaft may be greater than or equal to 10 mm, 20 mm, 30 mm, 40 mm, or 50 mm. As shown in the embodiment of FIG. 1B, hole depth D4 is the distance from the external tissue surface 162 to the tissue surface 160 internal to the patient. In some embodiments, a hole depth in a patient for insertion of the contacts and outer shaft may be less than or equal to 50 mm, 40 mm, 30 mm, 20 mm, or 10 mm. Combinations of the above-referenced ranges are also possible. For example, in some embodiments, a hole depth may be 10 to 50 mm, 20 to 40 mm, 20 to 30 mm, 30 to 40 mm, or any other suitable depth.

In some embodiments, a hole diameter in a patient for insertion of the contacts and outer shaft may be greater than or equal to 1 mm, 2 mm, 3 mm, 4 mm, 4.5 mm, 5 mm, 5.5 mm, 6 mm, 7 mm, 8 mm, 9 mm, 10 mm, 15 mm, 20 mm or greater. In some embodiments, a hole diameter may be less than or equal to 20 mm, 15 mm, 10 mm, 9 mm, 8 mm, 7 mm, 6 mm, 5.5 m, 5 mm, 4.5 mm, 4 mm, 3 mm, 2 mm, 1 mm, or less. Combinations of the above-referenced ranges are also possible. For example, in some embodiments, a hole diameter may be 1 to 20 mm, 1 to 15 mm, 1 to 10 mm, 1 to 9 mm, 1 to 8 mm, 2 to 7 mm, or 3 to 6 mm.

To allow for insertion of the outer shaft through a hole of any such size of the patient to reach a target tissue site, the outer shaft may be of any suitable dimensional parameters including an outer shaft length, denoted by D1 in FIG. 1A, as well outer and inner cross-sectional dimensions (e.g. outer diameter and inner diameter of a cylindrical outer shaft).

In some embodiments, an outer shaft length D1 may be less than or equal to 500 mm, 450 mm, 400 mm, 350 mm, 300 mm, 250 mm, 200 mm, 150 mm, 100 mm, 90 mm, 80 mm, 70 mm, 60 mm, 50 mm, 40 mm, 30 mm, or 20 mm. In some embodiments, the outer cross-sectional dimension (e.g. outer diameter, outer diagonal, etc.) of the outer shaft may be less than or equal to 15 mm, 14 mm, 13 mm, 12 mm, 11 mm, 10 mm, 9 mm, 8 mm, 7 mm, 6 mm or 5 mm. In some embodiments, the outer shaft may have a wall thickness of greater than or equal to 0.5 mm, 1 mm, 1.5 mm, 2 mm, or greater. While such outer shaft dimensions are disclosed, other dimensions may be used in other embodiments.

The outer shaft may be of a variety of suitable shapes to provide sufficient interior volume in the outer shaft to allow for one or more injection needles, among other components of a tissue injection device (e.g. the contacts in some embodiments), to be disposed within. In some embodiments, a suitable outer shaft shape may include, but is not limited to, circular cylinders, elliptical cylinders, prisms (e.g. rectangular prisms), cones, pyramids, or any other suitable shape. In some embodiments, an exterior surface of the outer shaft may be of a different shape than an interior surface of the outer shaft. For example, an outer shaft may include an exterior surface in the shape of a circular cylinder and an interior surface in the shape of an elliptical cylinder. While these arrangements are disclosed, the exterior and interior surfaces of the outer shaft may be of any suitable shape, size, or other characteristic as the disclosure is not so limited.

In some embodiments, the outer shaft is constructed and arranged to be rigid. Such a configuration may serve to ensure that the tissue injection device is stabilized and accurately positioned relative to the target tissue site upon engaging the target tissue surface. The outer shaft may also be constructed of a variety of suitable materials including plastics (e.g. polypropylene and polyethylene), metals (e.g. aluminum and stainless steel), or any other suitable material as the disclosure is not so limited.

FIG. 2A shows an enlarged view of the distal portion of the outer shaft 110 of FIG. 1A where the contacts 120 have been moved into the expanded configuration. As disclosed herein, the expanded configuration of the contacts may serve to provide a larger surface area configured to contact a corresponding tissue surface. In some embodiments, the contacts may provide one or more of the following advantages: increased stability of the tissue injection device against the tissue, accurate positioning of the tissue injection device on the tissue surface, and decreased amount of pressure subjected to the tissue, among other advantages disclosed above.

The one or more contacts may be of any suitable size, geometry, material, or other characteristic as needed to assist in stabilizing the injection device against a target tissue surface. In some embodiments, a suitable geometry of the contacts may include, but is not limited to rectangular prisms, pentagonal prisms, hexagonal prisms, cylinders, cones, and pyramids. In some embodiments, the contacts may be constructed of a material including, but not limited to plastics (e.g. polypropylene and polyethylene) and metals (e.g. aluminum and stainless steel).

The contacts may also have any number of curved, angled, flat, or otherwise shaped surfaces that engage with the tissue surface. For example, the base of the contacts may be flat to promote stability of the tissue injection device against tissue. In another example, one or more surfaces of the contacts may be angled to urge the contacts into the expanded configuration upon contacting the tissue surface, as is discussed in greater detail below.

In some embodiments, the one or more contacts may be constructed to be rigid and comprise any suitable number of pivoting members. The contacts may be disposed at the distal portion of the outer shaft such that they may be pivoted between contracted and expanded configurations. These pivoting contacts may be actuated between the contracted and expanded configurations using the actuator as disclosed herein.

In some embodiments, the one or more contacts may comprise any suitable number of flexible members, which may be constructed of a flexible (e.g. a hyperelastic) material. In some such embodiments, the contacts may be configured to be received within the interior volume of the outer shaft when in a contracted configuration. For example, the flexible contacts may be formed such that they are biased into the expanded configuration, but may be retained in the contracted configuration by being positioned within the outer shaft. In some embodiments, the outer shaft itself may also be retractable and extendable.

Such a configuration may permit the deployment of the one or more flexible contacts if the outer shaft were to be retracted relative to such contacts. Alternatively, the flexible contacts may be extended out of the shaft opening to deploy into the expanded configuration.

Any suitable number of contacts may be provided in a tissue injection device to promote stability and accurate positioning of the device relative to the target tissue surface. In some embodiments, a suitable number of contacts may be greater than or equal to 1, 2, 3, 4, 5, 6, 7, 8, 9, 10 or greater.

In some embodiments, a plurality of contacts may be provided and positioned at different angles relative to the outer shaft of the tissue injection device. For example, in an embodiment where three contacts are provided, the contacts may be arranged to contact a tissue surface such that the first, second, and third contacts are oriented at angles of 80 degrees, 90 degrees, and 100 degrees, respectively in relation to a longitudinal axis of the outer shaft. The inventors have recognized that the capability of the one or more contacts to be angled differently from one another relative to the outer shaft may serve to ensure that the tissue injection device is stabilized and accurately positioned on contoured tissue surfaces. Such a configuration may also serve to permit angled deployment of the injection needle into a target tissue site. While this specific arrangement is disclosed, any suitable number of contacts may be provided and differently angled from one another in any suitable manner to conform to the target tissue surface.

In some embodiments, a suitable angle of any number of the contacts provided in a tissue injection device oriented relative to the longitudinal axis of the outer shaft may be greater than or equal to 10 degrees, 20 degrees, 30 degrees, 40 degrees, 50 degrees, 60 degrees, 70 degrees, 80 degrees, 85 degrees, 90 degrees, 95 degrees, 100 degrees, 110 degrees, 120 degrees, 130 degrees, 140 degrees, 150 degrees, or greater.

In some embodiments, a plurality of contacts may each be positioned radially with respect to one another in any suitable manner. For example, if three contacts are provided, the contacts may each be spaced such that in the expanded configuration, the contacts are each separated by an angle of 120 degrees. In another example where three contacts are provided, the contacts may be spaced at different angles relative to one another. In such an example, the angle between the first and second contacts may be 90 degrees, the angle between the second and third contacts may be 120 degrees, and the angle between the third and first contacts may be 150 degrees. While these specific arrangements are disclosed, the contacts may be angled relative to one another in any suitable manner as the disclosure is not so limited.

FIG. 2B shows an enlarged view of the distal portion of the outer shaft 110 of FIG. 1A where the contacts 120 have been moved into the expanded configuration and an injection needle 130 has been actuated into the extended position. In the extended position, the injection needle 130 may be deployed past the distal portion opening of the outer shaft 110 at a distance D2. The distance D2 that the injection needle 130 is deployed may be controlled by an actuator as disclosed herein. In some embodiments, the contacts 120 may include angled surfaces that are configured to contact a corresponding tissue surface. The angled surfaces may be provided on any suitable region of the contacts as the disclosure is not so limited.

FIG. 2C shows an enlarged view of the distal portion of the outer shaft 110 of FIG. 1A where the contacts 120 have been moved into the expanded configuration and an injection needle 130 has been actuated into the extended position. This embodiment further shows the injection needle being inserted into a corresponding tissue surface 160.

The needle 130 may include one or more outlet openings 132 through which a therapeutic substance is released in order to introduce the substance into a target tissue site. The needle 130 may be hollow such that the therapeutic substance passes through a lumen of the needle and out through the outlet openings 132.

In some embodiments, the distance D2 that the needle is extended may be different than the insertion depth D3 of the needle into the corresponding tissue surface, as shown in FIG. 2C. In some embodiments, the insertion depth D3 is defined by the distance from a distal abutting surface 121 of the contact 120 when the contact is in an expanded configuration to the distal end 131 of the needle 130 when the needle 130 is in the extended configuration. In other embodiments, however, the distance of needle extension may be equivalent to the depth of needle insertion.

The distance D2 of the needle extension may also be of any suitable distance as the disclosure is not so limited. In some embodiments, a suitable distance D2 of the injection needle extension may be greater than or equal to 2 mm, 2.5 mm, 3 mm, 3.5 mm, 4 mm, 4.5 mm, 5 mm, 6 mm, 7 mm, 8 mm, 9 mm, 10 mm, 12 mm, 15 mm, 20 mm, or greater. In some embodiments, a suitable distance D2 of the injection needle extension may be less than or equal to 20 mm, 15 mm, 12 mm, 10 mm, 9 mm, 8 mm, 7 mm, 6 mm, 5 mm, 4.5 mm, 4 mm, 3.5 mm, 3 mm, 2.5 mm, 2 mm, 1.5 mm, 1 mm, 0.5 mm, or less.

The injection needle may be of any suitable size (e.g. needle length and diameter), shape (e.g. tapered tip or non-tapered tip), or other characteristic as the disclosure is not so limited. In some embodiments, the injection needle may have a diameter of at greater than or equal to about 0.05 mm, 0.1 mm, 0.15 mm, 0.2 mm, 0.25 mm, 0.3 mm, 0.35 mm, 0.4 mm, 0.45 mm, or 0.5 mm. In some embodiments, the injection needle may have a diameter of less than or equal to about 1 mm, 0.9 mm, 0.8 mm, 0.7 mm, 0.6 mm, 0.5 mm, 0.4 mm, 0.3 mm, 0.2 mm, 0.1 mm, or less. Combinations of the above referenced ranges are also possible. For example, in some embodiments, the injection needle may have a diameter of about 0.1 mm to 1 mm, 0.2 mm to 0.9 mm, 0.3 mm to 0.8 mm, 0.4 mm to 0.7 mm, or any other suitable combination of ranges.

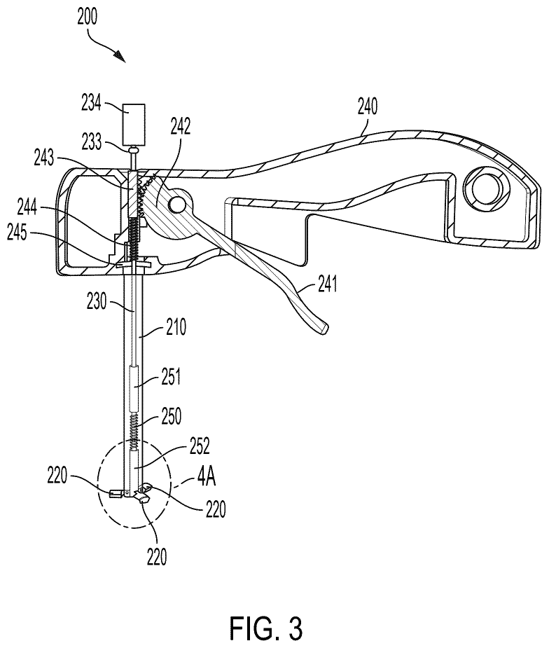

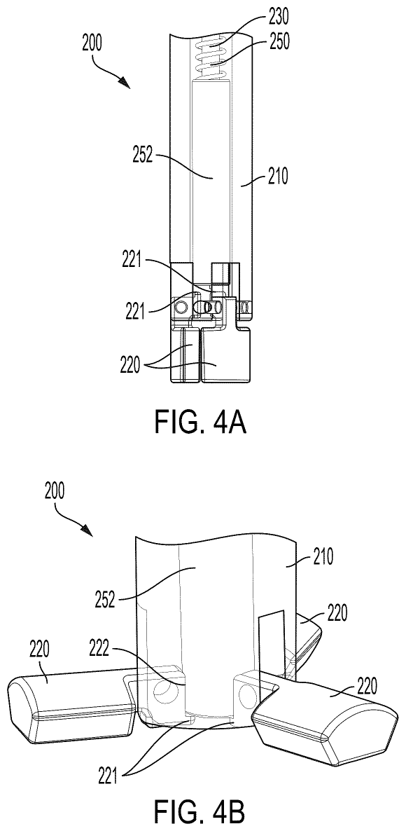

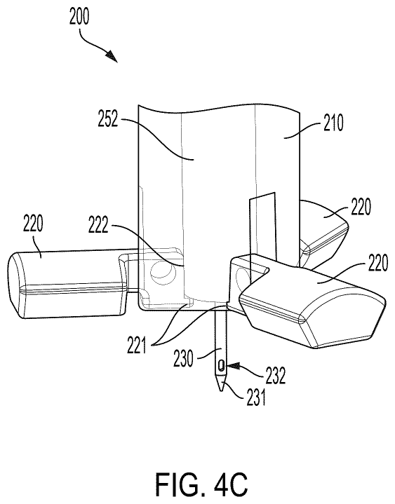

The illustrative embodiment of FIG. 3 and 4A-4C shows a tissue injection device 200 comprising an outer shaft 210 and contacts 220 disposed at the distal portion of the outer shaft 210. FIG. 3 additionally shows an injection needle 230 disposed within the outer shaft 210, and a device body 240 coupled to the proximal portion of the outer shaft 210. The device body 240 includes an actuator 241 in the form of a trigger. In some embodiments, the trigger 241 is used to actuate the injection needle 230 between the retracted and extended positions. The trigger 241 may also be used to actuate the contacts 220 between the contracted and expanded configurations. The trigger 241 includes a distal portion 242 having a plurality of teeth. The plurality of teeth of the distal portion 242 are configured to be interfaced with a plurality of teeth of a corresponding rack 243 affixed to the injection needle 230. Upon application of force to the trigger 241 by a user, the rack 243 is actuated in a deployment direction towards the distal portion of the outer shaft 210. Upon release of the application of force to trigger 241, an upper biasing member 244 disposed in the device body 240 biases the trigger 241 into a non-actuated state and in turn biases the injection needle 230 into the retracted position. The upper biasing member 244 is disposed between the rack 243 and a contact surface (e.g., a top portion 245 of the outer shaft 210) such that the upper biasing member 244 is compressed when force is applied to the trigger 241. The upper biasing member 244 may be at rest when the force is released from the trigger 241 and the trigger 241 returns to the non-actuated state.

The tissue injection device 200 further comprises a first cylindrical body 251 affixed to the injection needle 230, and an abutment body in the form of a second cylindrical body 252 surrounding a distal portion of the injection needle 230, where the second cylindrical body 252 is movable relative to the injection needle 230. In some embodiments, the first cylindrical body 251 may be fixed to the injection needle 230, e.g. adhesively attached, welded, integrally formed as a single piece at the same time (e.g. cast at the same time as a single piece), or any other suitable arrangement. A lower biasing member 250 (e.g. a spring) may be disposed between the first and second cylindrical bodies (251, 252). In some embodiments, the lower biasing member 250 may be attached to both the first and second cylindrical bodies (251, 252). The movement of the rack 243 in a deployment direction due to actuation of the trigger 241 moves both the injection needle 230 and the first cylindrical body 251 in the deployment direction towards the distal portion of the outer shaft 210. With the lower biasing member 250 connecting the first cylindrical body 251 to the second cylindrical body 252, movement of the first cylindrical body 251 in the deployment direction also causes the second cylindrical body 252 to move in the deployment direction.

As seen in FIG. 3, the tissue injection device 200 may include an inlet 233 (e.g., a luer fitting or other suitable inlet) configured to permit fluid communication with the lumen of the injection needle 230. Various therapeutic substances disclosed above may be introduced into the inlet 233. In some embodiments, a storage container 234, e.g. a vial, syringe, or other suitable container, may be used to introduce a therapeutic substance into the inlet 233. The storage container 234 may be coupleable with the inlet 133, directly or via connecting components such as tubing or other couplings.

FIG. 4A shows an enlarged view of the distal portion of the outer shaft 210 of FIG. 3, where the contacts 220 are in the contracted configuration. The contacts 220 include ridges 221 which are configured to be interfaced with the second cylindrical body 252. In the contracted configuration, the contacts 220 may be of a lesser transverse dimension than in the expanded configuration. In some embodiments, the contacts 220 and the outer shaft 210 may be of a substantially similar transverse dimension when the contacts are in the contracted configuration.

FIG. 4B shows an enlarged view of the distal portion of the outer shaft 210 of FIG. 3 where the contacts 220 are in the expanded configuration. With the second cylindrical body 252 being actuated using the mechanism described above in reference to FIG. 3, the second cylindrical body 252 abuts the ridges 221 of the contacts 220. As a result, the contacts 220 are pivoted from the contracted configuration shown in FIG. 4A into the expanded configuration shown in FIG. 4B. In some embodiments, the contacts 220 may include angled surfaces that are configured to contact a corresponding tissue surface. The angled surfaces may be provided on any suitable region of the contacts as the disclosure is not so limited.

In some embodiments, the presence of the ridges 221 may also help to prevent the second cylindrical body 252 from extending out of the outer shaft 210. The contacts 220 are prevented from further pivoting due to abutting of ends 222 against the side of the second cylindrical body 252, and thus the second cylindrical body 252 cannot move past the ridges 221. Such a configuration may also serve to provide a rigid stopping point of the contacts 220, where the contacts 220 may not extend past a predetermined position when in the expanded configuration due to contact with the second cylindrical body 252.

In some embodiments, the actuator (e.g. trigger mechanism) may be actuated such that the needle 230 is transitioned from the extended position to the retracted position. In reference to FIG. 3, movement of the needle 230 into the retracted position moves the cylindrical body 252 toward the retraction direction and out of abutment with the contacts 220, thereby permitting the contacts 220 to return to the contracted configuration. While this specific arrangement is disclosed, the actuator may be employed in any suitable arrangement to move the injection needle between a retracted and extended position and/or to move the one or more contacts between a contracted and expanded configuration.

In some embodiments, a trigger mechanism in the form of a rack and pinion type linear actuator may be employed to actuate the injection needle and/or one or more contacts as disclosed above. However, it should be appreciated that any suitable actuator may be used. For example, a suitable actuator may include a lever or a worm gear driven linear actuator.

FIG. 4C shows an enlarged view of the distal portion of the outer shaft 210 of FIG. 3 where the contacts 220 are in the expanded configuration and the injection needle 230 is in the extended position. The previously discussed lower biasing member 250 (see FIG. 3) connecting the first cylindrical body 251 and the second cylindrical body 252 may also permit relative movement between the two cylindrical bodies, thereby also permitting the needle 230 to move relative to and through the second cylindrical body 252 such that the needle 230 can be moved into the extended position. The lower biasing member 250 may be of a suitable stiffness such that the lower biasing member 250 may transmit force from the first cylindrical body 251 to the second cylindrical body 252 in order to turn the contacts 220 into the expanded configuration while also ensuring that the injection needle 230 is able to be actuated into the extended position past the distal end of the outer shaft 210.

The injection needle 230 includes a distal end 231 that may be tapered as shown in FIG. 4C, and the injection needle 230 may be advanced at a controlled depth into the target tissue site using suitable actuators as disclosed above. The injection needle 230 may include one or more outlet openings 232 through which a therapeutic substance is released in order to introduce the substance into a target tissue site. The injection needle 230 may be hollow such that the therapeutic substance passes through a lumen of the needle and out through the outlet openings 232.

In some embodiments, a tissue injection device may include one or more push wires that move contacts between contracted and expanded configurations.

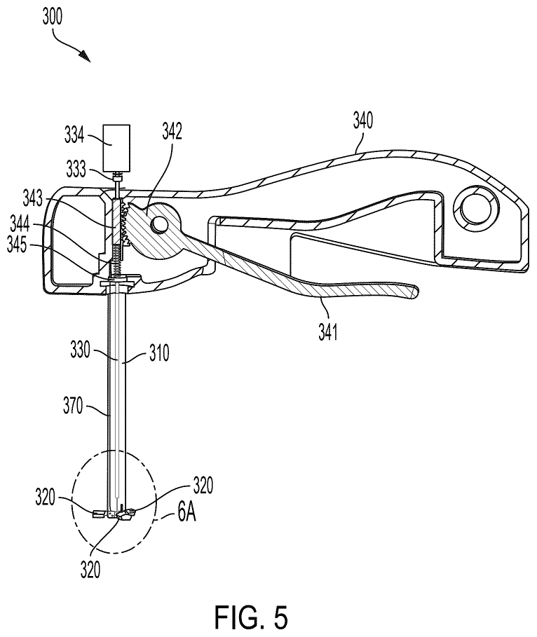

The illustrative embodiment of FIG. 5 shows a tissue injection device 300 comprising an outer shaft 310 and contacts 320 disposed at the distal portion of the outer shaft 310. FIG. 5 additionally shows an injection needle 330 disposed within the outer shaft 310, and a device body 340 coupled to the proximal portion of the outer shaft 310. The device body 340 includes an actuator 341 in the form of a trigger. In some embodiments, the trigger 341 is used to actuate the injection needle 330 between the retracted and extended positions. The trigger 341 may also be used to actuate the contacts 320 between the contracted and expanded configurations. The trigger 341 includes a distal portion 342 having a plurality of teeth. The plurality of teeth of the distal portion 342 are configured to be interfaced with a plurality of teeth of a corresponding rack 343 affixed to the injection needle 330. Upon application of force to the trigger 341 by a user, the rack 343 is actuated in a deployment direction towards the distal portion of the outer shaft 310. Upon release of the application of force to trigger 341, an upper biasing member 344 disposed in the device body 340 may bias the trigger 341 into a non-actuated state and may, in turn, bias the injection needle 330 into the retracted position. The upper biasing member 344 is disposed between the rack 343 and a contact surface 345 located at the top of the outer shaft 310 such that the upper biasing member 344 is compressed when force is applied to the trigger 341. The upper biasing member 344 may be at rest when the force is released from the trigger 341 and the trigger 341 returns to the non-actuated state.

The tissue injection device 300 further includes one or more push wires 370 that are configured to move the contacts 320 between the expanded and contracted configurations. Although the embodiment of FIG. 5 depicts only one push wire 370 for ease of illustration, it should be appreciated that each contact may have a dedicated push wire such that the tissue injection device may have a plurality of push wires. In other embodiments, any suitable number of push wires may be used, such as only a single push wire. In some embodiments, multiple contacts may share a single push wire, such that the total number of push wires may be smaller than the total number of contacts.

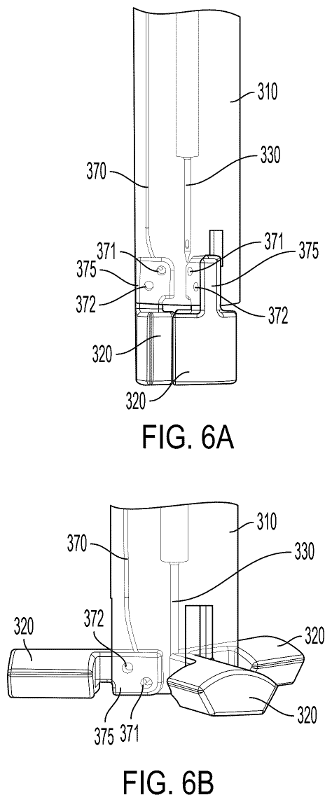

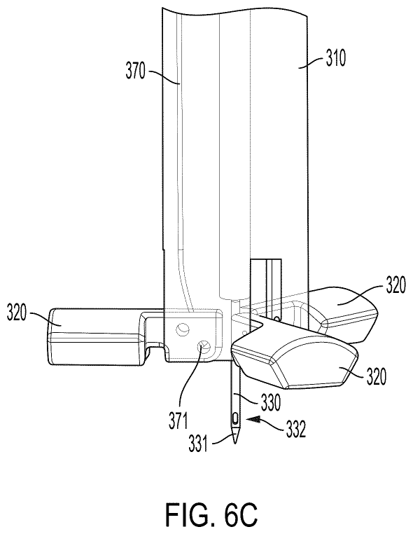

In some embodiments, a proximal end of the push wire may be coupled to the contact, e.g. by being affixed to the contact. As shown in FIGS. 6A and 6B, the push wire 370 is coupled to the contact 320. In some embodiments, each contact may have an extension portion 375, and the push wire may be coupled to the extension portion of the contact. In some embodiments, the push wire may be coupled to the contact by extending at least partially through an opening 371 in the contact 320. The push wire may be secured to the contact such that movement of the push wire 370 in turn moves the contacts 320 between expanded and contracted configurations.

As shown in FIGS. 6A and 6B, each contact 320 may have a pivot 372 about which the contact pivots. Movement of the push wire 370 in a deployment direction (the downward direction in FIG. 6A) may cause the contact 320 to pivot about the pivot 372 from the contracted configuration shown in FIG. 6A into the expanded configuration shown in FIG. 6B. Movement of the push wire in a retraction direction (the upward direction in FIG. 6B) may cause the contact 320 to pivot about the pivot 372 from the expanded configuration shown in FIG. 6B into the contracted configuration shown in FIG. 6A.

The push wires may move in a deployment direction and/or in a retraction direction in response to actuation of a device actuator 341. In some embodiments, actuation of the device actuator 341 (e.g., squeezing a trigger) may cause the push wires to move in a deployment direction, and releasing the actuator may cause the push wires to move back in a retraction direction. The push wires may be biased toward the retraction direction by, for example, the upper biasing member 344. In some embodiments, the one or more push wires 370 may be affixed directly or indirectly to the injection needle 330 or any other suitable portion of the tissue injection device 300 as the disclosure is not so limited.

In some embodiments, upon actuation of the trigger 341, both the injection needle 330 and the push wire 370 are actuated to move towards the distal end of the outer shaft 310. As a result, the distal end of the push wire applies a force to the contacts 320 such that the contacts 320 are rotated and deployed into the expanded configuration.

In some embodiments, a single push wire 370 may actuate all of the contacts 320 into the expanded configuration. In some such embodiments, each of the contacts 320 may be connected to one another via a suitable attachment (e.g., a ring), such that actuation of the one push wire 370 actuates all of the contacts 320. In other embodiments however, a single push wire 370 may only actuate the contact 320 that it is engaged to. Upon release of the trigger 341, the push wire 370 may be biased to move toward the proximal end of the outer shaft 310, and thereby the contacts 320 may be returned to the contracted configuration.

As noted above, a plurality of push wires may be used within a tissue injection device 300 rather than a single push wire 370. In some such embodiments, each of the plurality of push wires may have a distal end connected to the contacts via openings disposed in the contacts. Such an arrangement may allow each push wire to individually actuate a corresponding contact between the expanded and contracted configurations.

In FIG. 5, the tissue injection device 300 also includes an inlet 333, such as a luer fitting, configured to receive therapeutic substances into the device. The inlet 333 may be in fluid communication with a lumen of the injection needle 330. Various therapeutic substances disclosed above may be introduced into the inlet 333. In some embodiments, a storage container 334, e.g. a vial, syringe, or other suitable container, may be used to introduce therapeutic substances into the inlet 333. The storage container 334 may be coupleable with the inlet 333, directly or via connecting components such as tubing or other couplings.

In some embodiments, the push wires may be of any suitable size, stiffness, material, or other characteristic to permit sufficient force to be applied to the contacts such that the contacts pivot into the expanded configuration. In some embodiments, a suitable length of the push wires may be greater than or equal to 5 mm, 10 mm, 15 mm, 20 mm, 25 mm, 30 mm, 35 mm, 40 mm, 45 mm, 50 mm, or greater. In some embodiments, the push wires may be of a suitable material including, but not limited to stainless steel, aluminum, titanium, or any other suitable material as the disclosure is not so limited. The inventors have also recognized that, in some embodiments, it may be desirable for the push wires to be of a certain stiffness such that the push wires are able to apply a sufficient force to move the contacts into the expanded configuration while also being adequately flexible to permit the expansion of the contacts while the contacts are secured to the push wires.

FIG. 6A shows an enlarged view of the distal portion of the outer shaft 310 of FIG. 5 where the contacts 320 are in the contracted configuration. In the contracted configuration, the contacts 320 may be of a lesser transverse dimension than in the expanded configuration. In some embodiments, the contacts 320 and the outer shaft 310 may be of a substantially similar transverse dimension when the contacts are in the contracted configuration.

FIG. 6B shows an enlarged view of the distal portion of the outer shaft 310 of FIG. 5 where the contacts 320 are in the expanded configuration. As disclosed above, upon actuation of the trigger 341, the push wire 370 applies a force to the contacts 320, thereby resulting in the contacts 320 being deployed into the expanded configuration.

In some embodiments, the contacts 320 may include angled surfaces that are configured to contact a corresponding tissue surface. The angled surfaces may be provided on any suitable region of the contacts as the disclosure is not so limited.

FIG. 6C shows an enlarged view of the distal portion of the outer shaft 310 of FIG. 5 where the contacts 320 are in the expanded configuration and the injection needle 330 is in the extended position. The injection needle 330 includes a distal end 331 and may be advanced at a controlled depth into the target tissue site using the trigger 341 or other suitable actuators as disclosed above. The injection needle 330 includes one or more outlet openings 332 through which a therapeutic substance is released in order to introduce the substance into a target tissue site. The injection needle 330 may be hollow such that the therapeutic substance passes through a lumen of the needle and out through the outlet openings 332.

According to another aspect, a tissue injection device may include at least one stop surface disposed on a distal end of an outer shaft of the injection device. The tissue injection device may include one or more contacts disposed on the distal portion of the outer shaft that may be constructed and arranged to contact a tissue surface, as disclosed above. The contacts may also be moveable between a contracted and an expanded configuration, with the contacts being biased towards the contracted configuration. In some such arrangements, upon contacting the tissue surface, the contacts may be urged into the expanded configuration to provide a larger contact area to stabilize the tissue injection device. The at least one stop surface may be configured to abut the one or more contacts to prevent movement of the contacts beyond a predetermined position when transitioning into the expanded configuration.

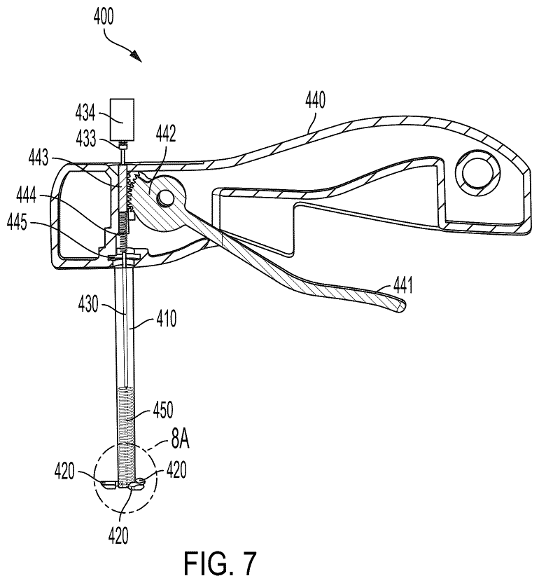

The illustrative embodiment of FIG. 7 shows a tissue injection device 400 comprising an outer shaft 410 and contacts 420 disposed at the distal portion of the outer shaft 410. FIG. 7 additionally shows an injection needle 430 disposed within the outer shaft 410, and a device body 440 secured to the proximal portion of the outer shaft 410. The device body 440 includes an actuator in the form of a trigger 441. The trigger mechanism functions similarly to the embodiments of FIGS. 3 and 5 to actuate the injection needle 430 into the extended position by interfacing teeth of a distal trigger portion 442 with a rack 443 affixed to the injection needle 430, thereby actuating the injection needle 430 in a deployment direction when a force is applied to the trigger 441 by the user. Upon release of the application of force to trigger 441, an upper biasing member 444 disposed in the device body 440 biases the trigger 441 into a non-actuated state and in turn biases the injection needle 430 into the retracted position. The upper biasing member 444 is disposed between the rack 443 and a contact surface (e.g., a top portion 445 of the outer shaft 410) such that the upper biasing member 444 is compressed when force is applied to the trigger 441. The upper biasing member 444 may be at rest when the force is released from the trigger 441 and the trigger 441 returns to the non-actuated state. In the embodiment of FIG. 7, the trigger mechanism may not actuate the contacts 420 between the expanded and contracted configurations. However, it should be appreciated that in other embodiments, the trigger mechanism may actuate the contacts between the expanded and contracted configurations.

In FIG. 7, the tissue injection device 400 also includes an inlet 433, such as a luer fitting, configured to received therapeutic substances into the device. The inlet 433 may be in fluid communication with a lumen of the injection needle 430. Various therapeutic substances disclosed above may be introduced into the inlet 433. In some embodiments, a storage container 434, e.g. a vial, syringe, or other suitable container, may be used to introduce therapeutic substances into the inlet 433. The storage container 434 may be coupleable with the inlet 433, directly or via connecting components such as tubing or other couplings.

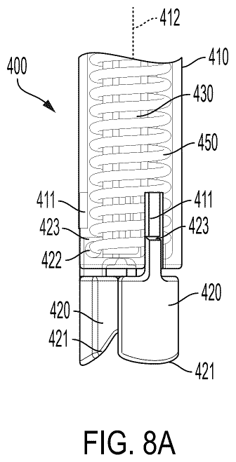

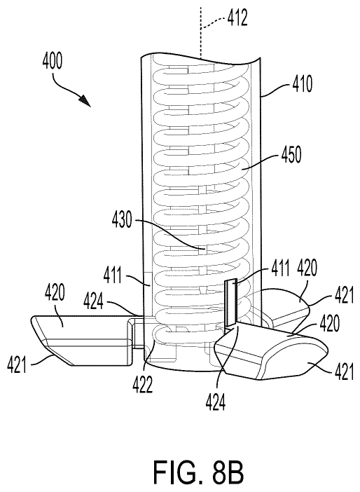

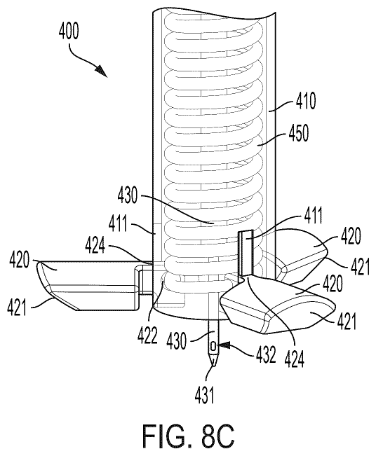

FIG. 8A shows an enlarged view of the distal portion of the outer shaft 410 of FIG. 7 where the contacts 420 are in the contracted configuration. The tissue injection device 400 comprises a lower biasing member 450 disposed within the outer shaft 410. In this arrangement, the lower biasing member 450 is threaded through passages 422 in the contacts 420, and the contacts 420 are biased by the lower biasing member 450 into the contracted configuration. In the contracted configuration, the contacts 420 may be of a lesser transverse dimension than in the expanded configuration. In some such arrangements, the contacts 420 and the outer shaft 410 may be of a substantially similar transverse dimension when the contacts are in the contracted configuration. In some embodiments, the contacts 420 may each include a first surface 423 which may contact the stop surfaces 411, which may therefore prevent movement of the contacts 420 past a predetermined position when in the contracted configuration.

In some embodiments, the contacts 420 may include angled surfaces 421 that are configured to facilitate contact with a corresponding tissue surface. Upon contacting the tissue surface, the angled surfaces 421 of the contacts 420 may facilitate sliding of the surfaces 421 against the tissue surface. As the device is pressed down onto the tissue, the normal force exerted by the tissue against the contacts 420 may overcome the biasing force of the biasing member 450, causing the contacts to slide outwardly (e.g. away from the longitudinal axis 412 of the outer shaft) and thereby move into the expanded configuration shown in FIG. 8B. While the contacts 420 are pressed down into contact with the tissue, the biasing member 450 may compress, storing potential energy. The angled surfaces 421 of the contacts 420 may be of any suitable angle as necessary to facilitate movement of the contacts 420 into the expanded configuration upon contacting a tissue surface. In some embodiments, a suitable angle of the angled surfaces relative to the tissue surface may be greater than or equal to about 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, or greater. In some embodiments, a suitable angle of the angled surfaces relative to the tissue surface may be less than or equal to about 75 degrees, 70 degrees, 65 degrees, 60 degrees, 55 degrees, 50 degrees, 45 degrees, 40 degrees, 35 degrees, 30 degrees, 25 degrees, 20 degrees, 15 degrees, 10 degrees, or less.

FIG. 8B shows an enlarged view of the distal portion of the outer shaft 410 of FIG. 7 where the contacts 420 are in the expanded configuration. In some embodiments, the distal portion of the outer shaft 410 may include stop surfaces 411. The stop surfaces 411 may be configured to abut a second surface 424 of the contacts 420 to restrict movement of the contacts 420 past a predetermined position when in the expanded configuration. As an example, in the illustrative embodiment of FIG. 8B, the stop surfaces 411 prevent the contacts 420 from rotating greater than 90 degrees from the longitudinal axis 412 of the outer shaft 410. However, it should be appreciated that in other embodiments, it should be appreciated that maximum rotation angles other than 90 degrees may be implemented.

In some embodiments, the stop surfaces may be oriented at different heights to permit the contacts to be differently angled with respect to one another. For example, three contacts may be provided, each having a stop surface. Each of the stop surfaces may be of a different height relative to one another such that the first, second, and third contacts are oriented at angles of 80 degrees, 90 degrees, and 100 degrees, respectively in relation to the longitudinal axis of the outer shaft when the contacts are in the expanded configuration. As disclosed above, this may allow for the contacts of the tissue injection device to conform to contours in the target tissue surface to promote stability of the injection device and/or to permit angled injection of the injection needle.

FIG. 8C shows an enlarged view of the distal portion of the outer shaft 210 of FIG. 7 where the contacts 420 are in the expanded configuration and the injection needle 430 is in the extended position. In some embodiments, the contacts 420 may be transitioned into the expanded configuration prior to transitioning the injection needle 430 into the extended position. Specifically, the angled surfaces 421 of the contacts 420 may contact the tissue surface and may move into the expanded configuration. Subsequently, actuation of the actuator (e.g. trigger 441) may cause the injection needle 430 to move into the target tissue site at a controlled depth. The injection needle 430 includes a distal end 431 and may include one or more outlet openings 432 through which a therapeutic substance is released in order to introduce the substance into a target tissue site. The injection needle 430 may be hollow such that the therapeutic substance passes through a lumen of the needle and out through the outlet openings 432. As discussed above, the contacts 420 may be biased to return to the contracted configuration due to the biasing member 450. When the force pressing the contacts 421 into contact with tissue ceases (e.g. a user stops pushing down the device onto the tissue), the biasing member 450 decompresses, releasing stored potential energy, causing the contacts 420 to rotate and return back to the contracted configuration.

In some embodiments, a tissue injection device may further comprise one or more imaging modalities. These imaging modalities may serve to provide imaging of the tissue injection site during operation of the injection device. The one or more imaging modalities may be integrated in the tissue injection device in any suitable manner. In some embodiments, the imaging modalities may be inserted through the outer shaft of the injection device and extend past the shaft opening to the tissue injection site. In some embodiments, the imaging modalities may be mounted to the exterior and/or interior of the outer shaft of the injection device. The imaging modalities may be of any suitable type as the disclosure is not so limited. In some embodiments, a suitable imaging modality may include visualization catheters such as flexible fiber optic scopes.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A tissue injection device comprising:
an actuator;
an outer shaft having a proximal portion and a distal portion, wherein the distal portion of the outer shaft includes a shaft opening;
one or more contacts constructed and arranged to contact a tissue surface, wherein the one or more contacts are moveable between a contracted configuration and an expanded configuration; and
an injection needle moveable through at least a portion of the outer shaft, the injection needle having a retracted position and an extended position,
wherein actuation of the actuator causes the injection needle to move between the retracted position and the extended position.

2. The tissue injection device of claim 1, wherein actuation of the actuator causes the one or more contacts to move between the contracted configuration and the expanded configuration.

3. The tissue injection device of claim 1, further comprising a stop surface at the distal portion of the outer shaft, the stop surface configured to abut against the one or more contacts to prevent movement of the one or more contacts beyond a predetermined position.

4. The tissue injection device of claim 1, further comprising an abutment body moveable between an unactuated position and an actuated position, wherein in the actuated position, the abutment body abuts against the one or more contacts to move the one or more contacts into the expanded configuration.

5. The tissue injection device of claim 4, wherein the abutment body comprises a cylinder.

6. The tissue injection device of claim 1, wherein the one or more contacts are biased toward the contracted configuration.

7. The tissue injection device of claim 6, wherein the one or more contacts are configured to move from the contracted configuration to the expanded configuration due to contact of the one or more contacts against tissue.

8. The tissue injection device of claim 7, wherein the one or more contacts include one or more angled surfaces configured to facilitate movement of the one or more contacts into the expanded configuration during contact of the one or more contacts against tissue.

9. The tissue injection device of claim 6, further comprising a biasing member configured to bias the one or more contacts toward the contracted configuration.

10. The tissue injection device of claim 1, further comprising one or more push wires, wherein the one or more push wires are configured to move the one or more contacts into the expanded configuration by way of rotating the one or more contacts around a pivot.

11. The tissue injection device of claim 1, wherein the outer shaft is rigid.

12-13. (canceled)

14. The tissue injection device of claim 1, further comprising a handle, wherein the actuator comprises a trigger on the handle.

15-17. (canceled)

18. The tissue injection device of claim 1, wherein the one or more contacts includes a plurality of contacts, and wherein at least one of the plurality of contacts is positioned at a different angle relative to the outer shaft than the other contacts when in the expanded configuration.

19. (canceled)

20. The tissue injection device of claim 1, wherein the needle is biased into the retracted position.

21. A tissue injection device comprising:
an outer shaft having a proximal portion and a distal portion, wherein the distal portion of the outer shaft includes a shaft opening;
one or more contacts configured to contact a tissue surface, wherein the one or more contacts are moveable between a contracted configuration and an expanded configuration, and wherein the one or more contacts are biased toward the contracted configuration;
an injection needle moveable through at least a portion of the outer shaft; and
at least one stop surface disposed at the distal portion of outer shaft, the at least one stop surface configured to abut against the one or more contacts to prevent movement of the one or more contacts beyond a predetermined position.

22. The tissue injection device of claim 21, further comprising an actuator, wherein actuation of the actuator causes the injection needle to move between a retracted position and an extended position.

23. The tissue injection device of claim 21, wherein the one or more contacts include one or more angled surfaces configured to facilitate movement of the one or more contacts into the expanded configuration during contact of the one or more contacts against tissue.

24. The tissue injection device of claim 21, wherein the outer shaft is rigid.

25-26. (canceled)

27. The tissue injection device of claim 22, further comprising a handle, wherein the actuator comprises a trigger on the handle.

28-29. (canceled)

30. The tissue injection device of claim 22, wherein the needle is biased into the retracted position.
